(19) United States
(12) Patent Application Publication
(10) Pub. No.: US 2011/0300596 A1
(43) Pub. Date: Dec. 8, 2011

(54) PREPARATION METHOD FOR ALCOHOL FROM CARBOXYLIC ACID BY ONE-STEP PROCESS

(75) Inventors: Seong Ho Lee, Daejeon (KR); Seung Hoon Oh, Seoul (KR); Young Seek Yoon, Gwangju (KR); In Ho Cho, Seoul (KR); Sin Young Kang, Daejeon (KR); Gi Ho Goh, Daejeon (KR); Cher Hee Park, Incheon (KR); Hee Soo Kim, Daejeon (KR)

(73) Assignee: SK INNOVATION CO., LTD., Seoul (KR)

(21) Appl. No.: 13/133,988

(22) PCT Filed: Dec. 10, 2009
(86) PCT No.: PCT/KR2009/007408

§ 371 Date: Aug. 25, 2011

(30) Foreign Application Priority Data
Dec. 12, 2008 (KR) 10-2008-0126542
Dec. 9, 2009 (KR) 10-2009-0121930

(51) Int. Cl. C12P 7/16; C07C 29/149

(52) U.S. Cl. 435/160; 568/885

(57) ABSTRACT

The present invention relates to a preparation method for alcohol by reacting carboxylic acid, alcohol, and hydrogen using hydrogenation catalysts. More specifically, the invention relates to a method for preparing alcohol by performing esterification and hydrocracking in a one-step process using hydrogenation catalysts instead of a two-step process. According to the invention, alcohol is prepared from carboxylic acid through esterification and hydrogenation in a one-step process using hydrogenation catalysts. Therefore, production costs and by-product treatment costs can be reduced in comparison to a two-step process. In addition, the invention is effective and economical since it can produce alcohol at relatively high yield by a simple process. Further, the invention allows high yield at relatively lower pressure when compared to alcohol production from carboxylic acid through hydrogenation without esterification and solves the problems of leaching by catalysts.

### TECHNICAL FIELD

The present invention relates to a method of preparing an alcohol by reacting a carboxylic acid, an alcohol, and hydrogen using a hydrogenation catalyst, and more particularly to a method of preparing an alcohol via esterification and hydrocracking in a one-step process using a hydrogenation catalyst in lieu of a two-step process.

### BACKGROUND ART

Typically, methods of preparing alcohol from carboxylic acid include a two-step process comprising adding an alcohol to a carboxylic acid so that the carboxylic acid is esterified and then adding hydrogen thereto so that hydrocracking occurs thereby obtaining an alcohol, and direct reduction comprising adding hydrogen to a carboxylic acid thereby obtaining an alcohol.

Preparation of an alcohol by adding hydrogen to a carboxylic acid requires high temperature and high pressure conditions and a noble metal catalyst, so that the alcohol is efficiently prepared from the carboxylic acid via esterification and hydrocracking.

US Patent Application No. 2008/0248540 discloses a method of synthesizing butanol by directly reacting butyric acid and hydrogen.

Typical preparation of alcohol from carboxylic acid via direct hydrogenation under high pressure conditions without esterification is problematic because of leaching of the catalyst used for the reaction, as well as the reaction under high pressure. For example, in the case where butyric acid is not esterified but is directly hydrogenated to prepare butanol, even when hydrogen under high pressure and an optimal noble metal catalyst are used, it is difficult to obtain a high yield of 90% or more, and also the metal component of the catalyst is directly exposed to butyric acid, undesirably facilitating the leaching of metal. Such leaching necessitates frequent replacement of the catalyst, undesirably increasing the butanol preparation cost.

An ester is a compound in the form of RCOOR′ made by eliminating water via a reaction between an organic carboxylic acid (RCOOH) and an alcohol (R′OH). In the case where the acid is acetic acid, it is provided in the form of CH₃COOCₙH₂ₙ₋₁ using methyl, ethyl, propyl, butyl, pentyl, etc., such as methyl acetate (CH₃COOCH₃) or ethyl acetate (CH₃COOC₂H₅). In the case where the alcohol is an aromatic or another type of compound, the molecular formula depends on the rule of the corresponding type. Even when various acids such as butyric acid, benzoic acid, and salicylic acid having an increased number of carbons or a changed structure are used in lieu of acetic acid, the structures thereof are also dependant on the above rule. After such esterification, hydrocracking is carried out, thereby attaining the corresponding alcohol.

For example, acetic acid is reacted with ethanol thus forming ethyl acetate ester, which is then hydrocracked to yield ethanol. These reactions are represented below.

CH₃COOH+C₂H₅OH-->CH₃COOC₂H₅+H₂O

CH₃COOC₂H₅+2H₂-->2C₂H₅OH

In the above reactions, esterification is carried out using a catalyst comprising sulfuric acid or an acidic resin in a batch reactor and then a hydrogenation catalyst is used so that ethanol is finally synthesized.

A typical method of preparing butanol from butyric acid includes the following reactions.

(1) Esterification

Butyric acid+butanol→butylbutyrate+H₂O, ΔH=−16.3 kJ/mole

(2) Hydrocracking

Butylbutyrate+2H₂→2BuOH, ΔH=−24.3 kJ/mole

Upon esterification, an acid ion exchange resin should be used as a catalyst. In order to increase the efficiency of the method, when not a batch reactor but a continuous reactor is used, it is difficult to load the ion exchange resin into the packed bed of the reactor because of muddy properties of the ion exchange resin. Furthermore, leaching of the ion exchanged component of the ion exchange resin may occur.

Because esterification has an equilibrium conversion depending on the reaction conditions, it is important that reaction conditions adapted for a high equilibrium conversion be imparted in order to increase a butanol yield. To this end, a specific component (e.g. butanol) in a feed is typically reacted in an excessive amount. In the case where the conversion of feed is not high, the product contains unreacted butyric acid, and butyric acid should be undesirably separated and nocovered from the final product, namely, butanol.

After esterification, hydrocracking becomes favorable in proportion to increases in hydrogen flow rate and pressure.

A typical two-step process for producing alcohol from carboxylic acid requires respective catalysts for esterification and hydrocracking, and there are many cases where the esterified intermediate compound should be additionally separated, and the method becomes complicated.

On the other hand, production of butyric acid using microorganism fermentation includes using strains such as Clostidium tyrobutyricum or Clostridium acetobutyrictum, and a lot of effort for more producibly developing strains is ongoing. Further, various carbon sources are employed as the carbon supply source of strains.

Moreover, a variety of attempts are being made to efficiently extract butyric acid from a fermentation broth and to perform liquid-liquid extraction using an insoluble organic solvent. Hence, there are proposed methods of obtaining butanol, by recovering butanol from a fermentation broth using a specific solvent having a high butanol extraction coefficient, recovering butanol using a difference in boiling point between the solvent and butanol, and regenerating the solvent.

U.S. Pat. No. 4,260,836 discloses a liquid-liquid extraction method from a fermentation broth using a fluorocarbon having a high butanol extraction coefficient, and U.S. Pat. No. 4,628,116 discloses a method of liquid-liquid extracting butanol and butyric acid from a fermentation broth using a vinyl bromide solution.

### DISCLOSURE

### Technical Problem

With the goal of solving the above problems, an object of the present invention is to provide a method of preparing an alcohol via a one-step process, in lieu of a two-step process of esterification and hydrocracking or direct reduction of carboxylic acid into alcohol.

Another object of the present invention is to provide a method of efficiently preparing an alcohol from a carboxylic acid by extracting the carboxylic acid from a microorganism fermentation broth.

The technical problems that are intended to be resolved in the present invention are not limited to the above objects, and other technical problems will be able to be understood by a person of ordinary skill in the art from the following description.

### Technical Solution

In order to accomplish the above objects, an aspect of the present invention provides a method of preparing an alcohol in a one-step process by reacting a carboxylic acid, an alcohol, and hydrogen, using a hydrogenation catalyst.

In this aspect, a C2˜10 alkyl carboxylic acid, a C3˜10 cycloalkyl carboxylic acid, a C6˜10 aromatic carboxylic acid, or mixtures thereof may be applied to the one-step process.

In this aspect, the carboxylic acid such as acetic acid, propionic acid, butyric acid, pentanoic acid, hexanoic acid, or mixtures thereof may be applied to the one-step process.

In this aspect, the alcohol including a C2˜10 alcohol, a C3˜10 cycloalkyl alcohol, a C6˜10 aromatic alcohol, or mixtures thereof may be applied to the one-step process.

In this aspect, the alcohol such as ethanol, propanol, butanol, pentanol, hexanol, or alcohol mixtures thereof may be applied to the one-step process.

In this aspect, a C2˜10 alcohol including ethanol or butanol or may be produced from carboxylic acid contained in a microorganism fermentation broth.

In this aspect, the alcohol that is added to the carboxylic acid may be obtained by recirculating the alcohol prepared from the carboxylic acid.

In this aspect, in the one-step process for preparing alcohol from carboxylic acid, the molar ratio of alcohol to carboxylic acid may be 1.0 or more.

In this aspect, hydrogen may be supplied at a molar ratio of 1˜50 to the carboxylic acid, and hydrogen pressure may fall in a range of atmospheric pressure ˜100 bar.

In this aspect, the catalyst used in the one-step process for preparing alcohol from carboxylic acid may be a hydrogenation catalyst.

In this aspect, the hydrogenation catalyst may be a metal or a metal oxide, and specifically may include one or more selected from among Cr, Mn, Fe, Co, Ni, Cu, Zn, Al, Si, Mo, W, Pt, Pd, Ru, Re, Rh, Ag, Ir, and Au.

Another aspect of the present invention provides a method of preparing butanol, comprising supplying a carbohydrate so that butyric acid is fermented from a microorganism, extracting butyric acid from a fermentation broth, and reacting the extracted butyric acid with butyric acid, butanol, and hydrogen, using a hydrogenation catalyst.

In this aspect, the method may include esterification and hydrocracking, which are carried out in a one-step process.

In this aspect, extracting the butyric acid from the fermentation broth may comprise extracting the butyric acid using liquid-liquid extraction.

In this aspect, extracting the butyric acid may further comprise distilling an extraction solvent from the extracted butyric acid.

In this aspect, the molar ratio of butanol to butyric acid may be 1.0˜50.

In this aspect, hydrogen may be supplied at a molar ratio of 1˜50 to butyric acid, and hydrogen pressure may fall in a range of atmospheric pressure ˜100 bar.

In this aspect, the hydrogenation catalyst may be a metal or a metal oxide.

In this aspect, the hydrogenation catalyst may be one or more selected from among Cr, Mn, Fe, Co, Ni, Cu, Zn, Al, Si, Mo, W, Pt, Pd, Ru, Re, Rh, Ag, Ir, Au, and metal oxides thereof.

The specific contents of other aspects or embodiments of the present invention are included in the following detailed specification.

### Advantageous Effects

According to the present invention, an alcohol can be prepared from a carboxylic acid via esterification and hydrogenation in a one-step process using a hydrogenation catalyst, thus reducing the production cost or the byproduct treatment cost, compared to when using a two-step process. Further, an alcohol can be produced in a comparatively high yield using a simple process, thus increasing preparation efficiency and generating economic benefits. Furthermore, an alcohol can be obtained in a high yield from a carboxylic acid at a comparatively lower pressure than in direct reduction of carboxylic acid into alcohol without esterification, and leaching problems of the catalyst can be resolved.

### DESCRIPTION OF DRAWINGS

FIG. 1 shows esterification and hydrocracking of butyric acid according to a conventional technique;

FIG. 2 shows a process of preparing butanol using a one-step process according to the present invention;

FIG. 3 schematically shows a micro-scale catalyst evaluation device;

FIG. 4 shows results of esterification of butyric acid and butanol using a hydrogenation catalyst.

FIG. 5 shows simultaneous reaction results of esterification and hydrocracking of butyric acid using a hydrogenation catalyst;

FIG. 6 shows simultaneous reaction results of esterification and hydrocracking depending on the temperature;

FIG. 7 shows the effects of the kind of reaction gas and pressure on simultaneous reactions of esterification and hydrocracking;

FIG. 8 shows a hydrocracking product using a hydrogenation catalyst, and simultaneous reaction products of esterification and hydrocracking, which are (a) a butylbutyrate hydrocracking product (FIG. 1), (b) an esterification-hydrocracking simultaneous reaction product (hydrogen at 10 bar) (FIG. 7), (c) an esterification-hydrocracking simultaneous reaction product (hydrogen at 30 bar) (FIG. 7), and (d) esterification-hydrocracking simultaneous reaction product (nitrogen at 30 bar) (FIG. 7);

FIG. 9 shows results of direct hydrogenation (reduction) of butyric acid; and

FIG. 10 shows simultaneous reaction results of esterification and hydrocracking of a teed mixture comprising butyric acid and acetic acid.

### BEST MODE

Hereinafter, a detailed description will be given of the present invention.

The present invention pertains to a method of preparing an alcohol by adding an alcohol, hydrogen, and a hydrogenation catalyst to a carboxylic acid.

In the method according to the present invention, an alcohol can be prepared from a carboxylic acid via esterification and hydrocracking in a one-step process in lieu of a two-step process. In such a one-step process, esterification and hydrocracking are simultaneously carried out so that an alcohol can be obtained.

The one-step process according to the present invention is schematically described below.

In the one-step process according to the present invention, butylbutyrate is continuously removed, and butanol is richly provided, so that equilibrium is shifted toward the forward reaction of esterification in accordance with the principle of Le Chatelier, thus maximizing the equilibrium conversion of esterification. When the equilibrium conversion is maximized in this way, 100% butyric acid can be reacted, and a resulting product contains no unreacted butyric acid, advantageously obviating the need to separate butyric acid from produced butanol.

Generally, metal dissolves well in an acid. Hence, when a feed is an acid, it is difficult to use a metal catalyst. Thus, a hydrogenation catalyst cannot be used despite having esterification reactivity.

However, in the one-step process according to the present invention, because esterification and hydrocracking are simultaneously carried out, leaching of metal is minimized. Specifically, butanol, which is one of feeds for esterification, is continuously produced by simultaneous reactions and thus participates as a feed for esterification, and thus the reaction rate is accelerated in proportion to an increase in the concentration of the feed. When the reaction rate of ester is accelerated in simultaneous reactions in this way, butyric acid may be rapidly removed, thus suppressing leaching of the catalyst.

As well, in the one-step process according to the present invention, an exothermic reaction of esterification and an exothermic reaction of hydrogenation are combined, thus increasing heat concentration effects, so that external heat supply may be reduced, thereby decreasing the preparation cost.

Also, the one-step process according to the present invention may be applied not only to batch reaction but also to continuous reaction.

In the present invention, the carboxylic acid includes a C2˜10 alkyl carboxylic a C3˜10 cycloalkyl carboxylic acid, a C6˜10 aromatic carboxylic acid, or mixtures thereof. In this one-step process, an alcohol may be prepared using a carboxylic acid mixture, instead of a single carboxylic acid. For example, acetic acid, propionic acid, butyric acid, pentanoic acid, hexanoic acid or mixtures thereof may be reacted with respective alcohols corresponding thereto or alcohol mixtures and with hydrogen using a hydrogenation catalyst, thus preparing alcohol.

According to the present invention, depending on the kind, amounts, and mixing ratio of carboxylic acid and alcohol used as feeds, the yield and the composition ratio of an alcohol product may be adjusted. Thus, the amount and the kind of alcohol feed or the mixing ratio of alcohol mixture may be adjusted so as to be adapted for the composition and the properties of a desired alcohol product, thereby optimizing operating conditions.

In the reaction for producing the alcohol horn the carboxylic acid, the reaction mechanism for synthesizing alcohol via esterification and hydrocracking is similar.

In the present invention, the alcohol feed used to convert carboxylic acid into alcohol includes a C2˜10 alkyl alcohol, a C3˜10 cyclo alcohol, a C6˜10 aromatic alcohol, or alcohol mixtures thereof.

In the present invention, for an acid mixture of two or more among a C2˜10 alkyl carboxylic acid, a C3˜10 cycloalkyl carboxylic acid, and a C6˜10 aromatic carboxylic acid, a single alcohol or an alcohol mixture of two or mom among a C2˜10 alkyl alcohol, a C3˜10 cyclo alcohol, and a C6˜10 aromatic alcohol may be used as a feed, thus preparing an alcohol mixture thereof.

In the present invention, the carboxylic acid may include acetic acid, propionic butyric acid, pentanoic acid (or valeric acid), hexanoic acid (or caproic acid), or mixtures thereof and a single alcohol or an alcohol mixture of two or more among ethanol, propanol, butanol, pentanol, and hexanol, corresponding to the product of carboxylic acid, is used as a feed, thus preparing ethanol, propanol, butanol, pentanol, hexanol, or alcohol mixtures thereof.

For example, in the case where an acid mixture of acetic acid and butyric acid is used as a feed, one selected from among ethanol and butanol, which are products thereof, may be re-used as a feed, or an alcohol mixture of two may be re-used as a feed, thus preparing an alcohol mixture of ethanol and butanol.

Also, the alcohol preparation process according to the present invention may be applied to a carboxylic acid-containing material, without limitation, and particularly a carboxylic acid-containing microorganism fermentation broth.

The alcohol prepared in the present invention includes a C2˜10 alcohol, a C3˜10 cycloalkyl alcohol, a C6˜10 aromatic alcohol, or alcohol mixtures thereof.

In the present invention, the alcohol added to the carboxylic acid is obtained by recirculating the alcohol prepared from the carboxylic acid. When the prepared alcohol is recirculated, the reverse reaction of esterification for producing butylbutyrate from butyric acid may be suppressed, thus maximizing the reaction yield for producing alcohol. Typically, esterification is a reversible reaction in which a forward reaction and a reverse reaction take place at the same time. As such, when the reverse reaction is suppressed by removing the alcohol that is produced via hydrocracking of the ester product, the forward reaction may be predominantly carried out.

In the method of preparing an alcohol via the reaction of carboxylic acid, alcohol, and hydrogen using a hydrogenation catalyst according to the present invention, for example, in the preparation of butanol by adding butanol to butyric acid, the molar ratio of butanol to butyric acid is 1.0˜50, particularly 2.0˜50. If the number of moles of butanol is increased, the reaction favorably occurs, but the molar ratio may be set within a range that does not negatively affect the recovery of butanol and recirculating it. If the molar ratio of butanol is less than 2.0, the concentration of butyric acid in the feed is comparatively increased, and thus the metal component of the catalyst may dissolve in butyric acid, undesirably contaminating the product and also shortening the lifetime of the catalyst. In particular, when the reaction starts, the reaction may become non-uniform and thus the catalyst may dissolve in butyric acid. It is preferred that the molar ratio of butanol to butyric acid be set to 2.5 or more and then decreased to 2.0 after stabilization of the reaction.

The reaction becomes favorable in proportion to increases in the flow rate of hydrogen added to butyric acid and in the pressure. If the flow rate of hydrogen is low, comparatively high pressure is required. The hydrogen pressure may range from atmospheric pressure to 100 bar, and hydrogen is supplied at a molar ratio of 1˜50, in particular, 10˜20, to butyric acid. When hydrogen is supplied at a molar ratio of 15 to butyric acid, the hydrogen pressure may be 30 bar.

In the present invention, the reaction temperature is 100˜300□. If the temperature is too low, the reaction rate is decreased and thus unreacted butyric acid and butylbutyrate may be produced, undesirably lowering the butanol yield. In contrast, if the temperature is too high, side reactions may occur and thus selectivity for butanol is decreased and the amount of impurities is increased, undesirably lowering the butanol yield and negatively affecting the purification of product. The reaction temperature is desirably set in the range of 150˜250□. However, when the reaction starts, it may non-uniformly occur, so that the metal component of the catalyst may dissolve in butyric acid, which may more easily take place at a temperature that is too low or too high. So, it is preferred for the reaction to start at 175□ and then be maintained at 200□ idler being stabilized.

In the case where an alcohol is prepared from a carboxylic acid contained in the microorganism fermentation broth, hydrogen that is added to the carboxylic acid of the fermentation broth may be used by recirculating a biogas produced from the microorganism fermentation broth.

Also in the case where butanol is prepared from butyric acid contained in the microorganism fermentation broth, hydrogen added to butyric acid of the fermentation broth may be used by recirculating a biogas produced from the microorganism fermentation broth, and hydrogen may be used in such a way that the biogas is directly used or hydrogen is additionally separated from the biogas.

The hydrogenation catalyst used in the present invention is provided in the form of one or more metals or metal oxides being supported on a support, and the metal or metal oxide supported on the catalyst may include one or more selected from among Cr, Mn, Fe, Co, Ni, Cu, Zn, Al, Si, Mo, W, Pt, Pd, Ru, Re, Rh, Ag, Ir, Au, and metal oxides thereof.

The support of the catalyst used in the present invention may include, but is not limited to, carbon, silica, alumina, etc. The support may further include a typical support depending on the predetermined purpose.

In a two-step process of esterification and hydrocracking for producing butanol from butyric acid of the fermentation broth, a resin catalyst may be used upon esterification. As such, however, in order to ensure thermal stability of the resin catalyst, the reaction temperature cannot be increased to 160° C. or higher though varying depending on the kind of resin catalyst, undesirably lowering the reaction rate to thereby increase the volume of esterification catalyst and the volume of reactor.

In a one-step process, a catalyst reactor the total volume of which is comparatively small may be used and the reaction heat generated upon two reactions may be utilized, advantageously reducing the external heat supply thanks to heat concentration effects, compared to the two-step process.

Further, in the case of the catalyst in the one-step process, an ion exchange resin catalyst for esterification is not additionally required as in the two-step process, and esterification and hydrocracking may be simultaneously achieved using the hydrogenation catalyst.

The method of preparing butanol using the one-step process is described below, which includes producing butyric acid in the microorganism fermentation broth, extracting butyric acid from the fermentation broth, and subjecting the extracted butyric acid to esterification and hydrocracking in the one-step process.

As shown in FIG. 2, the method according to the present invention includes extract fermentation, esterification, and hydrocracking, and specifically, fermentation, extraction, distillation, and a one-step process. In the present invention, hydrogen gas produced in the fermentation process is utilized for hydrocracking, and butanol resulting from hydrocracking is used for esterification, thus using the preparation efficiency.

In the present invention, a fermentation reactor is packed with a carrier having an immobilized strain for producing butyric acid, and a carbohydrate aqueous solution is continuously fed thereto, thus fermenting butyric acid.

The carbohydrate used for fermenting butyric acid in the present invention includes glucose, hexose, or pentose, and as well monosaccharides obtained by hydrolyzing polysaccharides. The carbohydrate is not particularly limited, and may further include a typical carbohydrate depending on the predetermined purpose.

The strain for fermenting the carbohydrate aqueous solution to produce butyric acid includes but is not limited to Clostridium tyrobutyricum or Clostridium butyricum, and may further include a typical microorganism depending on the predetermined purpose.

The strain for producing butyric acid is provided in the form of immobilized to the carrier in the reactor, and the carrier for immobilizing the strain may include a porous polymer carrier composed of polyurethane taking into consideration the stability of immobilization.

When the carbohydrate is fermented by the strain such as Clostridium tyrobutyricum, butyric acid is produced together with a biogas such as carbon dioxide and hydrogen gas. The biogas produced in the course of fermenting butyric acid has a composition of hydrogen and carbon dioxide at a volume ratio of about 1:1, and contains moisture of about 30 g/m³ corresponding to saturated vapor pressure at 30□ which is a fermentation temperature.

The biogas is introduced to a pressure swing adsorption unit from the fermentation reactor so that it is separated into hydrogen and carbon dioxide. A process of primarily removing contained moisture may be further added by disposing a dehydration pretreatment adsorption column (which is a water trap) upstream of the pressure swing adsorption unit, as necessary.

Although both the adsorption and membrane separation may be applied to easily separate hydrogen and carbon dioxide of the gas mixture, pressure swing adsorption is cost-effective because it can reduce the investment cost compared to the membrane separation requiring a large-scale membrane module.

In the method according to the present invention, the pressure swing adsorption unit includes a water trap using a silica, alumina or carbonaceous adsorbent and two or more adsorption columns packed with an adsorbent in multiple layers which is composed of one or a mixture of two or more selected from among zeolite A, zeolite X, zeolite Y and carbonaceous adsorbents. The adsorption pressure is set in the range of 2˜15 atm, particularly 5˜12 atm, and the desorption pressure is atmospheric pressure, and room-temperature operation is desirable.

In the present invention, the pressure swing adsorption unit for adsorption separating the gas mixture of hydrogen/carbon dioxide/moisture is operating at a pressure of about 10 bar. As such, the obtained 10 bar hydrogen may be used unchanged for subsequent hydrocracking, without additional pressurization.

On the other hand, the fermentation broth containing butyric acid obtained via fermentation is fed into a liquid-liquid extraction column to separate butyric acid, and trialkylamine insoluble in water is used as the extraction solvent in the liquid-liquid extraction column, and butyric acid is bonded with trialkylamine and thus converted into trialkylammonium butyrate which is then extracted.

Useful as the extraction solvent, trialkylamine include tripentylamine, trihexylamine, trioctylamine, tridecylamine, etc., which are insoluble in water. The extraction solvent is not limited the etc., and may further include a typical extraction solvent depending on the predetermined purpose.

Meanwhile, mono-amine or di-amine may produced into amide in the course of extraction and recovery, and thus is not used in the method according to the present invention.

The extract passed through the liquid-liquid extraction column includes trialkylamine as the extraction solvent and trialkylammonium butyrate converted from butyric acid, which are mixed together. When this extract is then introduced into a distillation column, trialkylammonium butyrate is decomposed into butyric acid and trialkylamine, thus obtaining butyric acid from the top of the distillation column and recovering trialkylamine from the bottom of the distillation column. The operating temperature of the distillation column may slightly vary depending on the kind of trialkylamine used as the extraction solvent. In the case of tripentylammonium butyrate produced using tripentylamine as the extraction solvent, decomposition begins at a temperature of 90˜100□. As such, trialkylamine recovered from the bottom of the distillation column may be re-used by being fed to the liquid-liquid extraction column as the extraction solvent for liquid-liquid extracting butyric acid as mentioned above.

In order to increase separation efficiency upon liquid-liquid extraction of butyric acid, a mixture of trialkylamine and a co-solvent such as diisopropylketone may be used as an extraction solvent, but the present invention is not limited thereto, and a typical co-solvent may be further included depending on the predetermined purpose.

Furthermore, butyric acid separated from the top of the distillation column is introduced along with butanol into a reactor where esterification and hydrocracking are simultaneously carried out, and is thus converted into butanol. As such, butanol used for the reaction may be used by recirculating butanol produced in the corresponding one-step process. The one-step process in which esterification and hydrocracking are simultaneously carried out is as described above.

### Mode or Invention

The following examples are set forth to illustrate the present invention, but are not to be construed as limiting the present invention, and may provide a better understanding of the present invention.

### Comparative Example 1

### Esterification of Butanol and Butyric Acid

Each of two tube-type glass reactors having an inner diameter of 12 mm was packed with 80 cc of Amberlyst-121wet available as a strong acid ion exchange resin from ROHM & HAAS, after which the inner temperature of the reactor was maintained at 110□.

For 10 hours from 5 hours after a feed comprising butyric acid and butanol mixed at a molar ratio of 1:2 began to pass through the reactor at a rate of 100 g/h, a reaction product and water produced during the reaction were collected, and 920 g of the reaction product and 75 g of water were obtained.

The results of analyzing the composition of the collected product and water showed that a butyric acid conversion was 98% or more and water produced by esterification contained 3.3% of butanol and 0.2% of butyric acid.

### Comparative Example 2

### Hydrocracking of Butylbutyrate

In the present invention, Katalco 83-3M commercially available from Jonson Mathey was used as a hydrogenation catalyst. A commercially available catalyst (CuZnOx/gamma-alumina, CuO: 51 wt %, ZnO: 31 wt %, alumina: the remainder) for converting aqueous gas was milled, and the catalyst, which passed through a 16 mesh sieve and was filtered on a 40 mesh sieve, was gathered at a volume of 12.0 cc and then loaded into a continuous tube-type reactor having an inner diameter of 10 mm. In order to pre-treat the catalyst, the catalyst was reduced with 5 vol % of a gas mixture of hydrogen and nitrogen at 200□ for 3 hours. Subsequently, butylbutyrate and hydrogen were supplied at 1.8 cc/h and 10 L/h respectively, the temperature of the catalyst bed was 150□, the downstream pressure of the reactor was maintained at 10 bar, and the feed was introduced up-flow.

After the temperature of the catalyst bed arrived at the normal level, the liquid product was gathered three times at intervals of 6 hours, and the product was analyzed using a gas chromatography (GC) unit [Hewlett Packard Co., HP5890 series] equipped with a polyethyleneglycol column (FLP-INNOWax column, 50 m×0.2 mm, 0.4 mm) and a flame ionization detector (FID). The average values of analyzed results are shown in Table 1 below. Also, the temperature of the catalyst bed was changed to 175□ or 200□ and the same test as above was performed. The results are summarized in Table 1 below.

TABLE 1

| Temp. of Catalyst Bed (° C.) | 150 | 175 | 200 |
|---|---|---|---|
| Conversion of Butylbutyrate (%) | 85 | 93 | 95 |
| Selectivity for butanol (%) | 99.9 | 99.8 | 99.7 |

### Comparative Example 3

### Direct Hydrogenation Reactivity of Butyric Acid Using Hydrogenation Catalyst

The reaction was performed under conditions in which the catalyst used, in-situ reduction of the catalyst just before the reaction, and the analysis method were the same as in Comparative Example 2, with the exception that butyric acid was supplied at 0.2 cc/min and the molar ratio of hydrogen to butyric acid was 15.

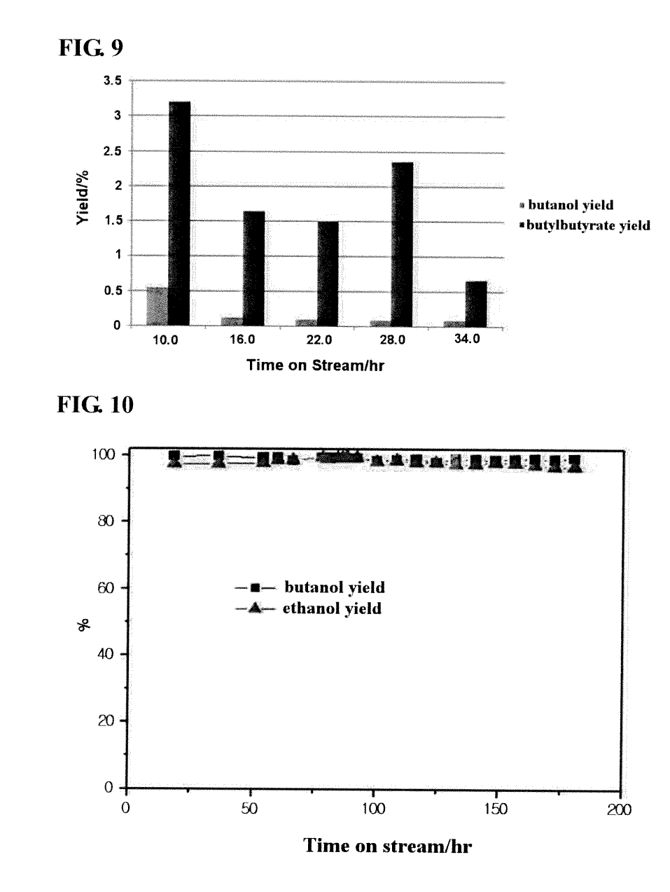

As shown in FIG. 9, the product yield was remarkably decreased by the direct hydrogenation of butyric acid and the product was composed mainly of butylbutyrate. In order to increase the yield, a considerably high pressure and a noble metal catalyst having high performance are required.

### Example 1

### Preparation of Butylbutyrate from Butyric Acid and Butanol

Butylbutyrate was prepared from butyric acid and butanol using a hydrogenation catalyst.

The reaction was performed under conditions in which the catalyst used, the reaction pressure, in-situ reduction of the catalyst just before the reaction, and the analysis method were the same as in Comparative Example 2, with the exception that a mixture of butyric acid and butanol was used as a feed, the molar ratio of butanol to butyric acid was 0.5˜1.0, the flow rate of the mixture was 0.1˜0.2 cc/min, the molar ratio of hydrogen to butylbutyrate was 15, and the reaction temperature was 175□.

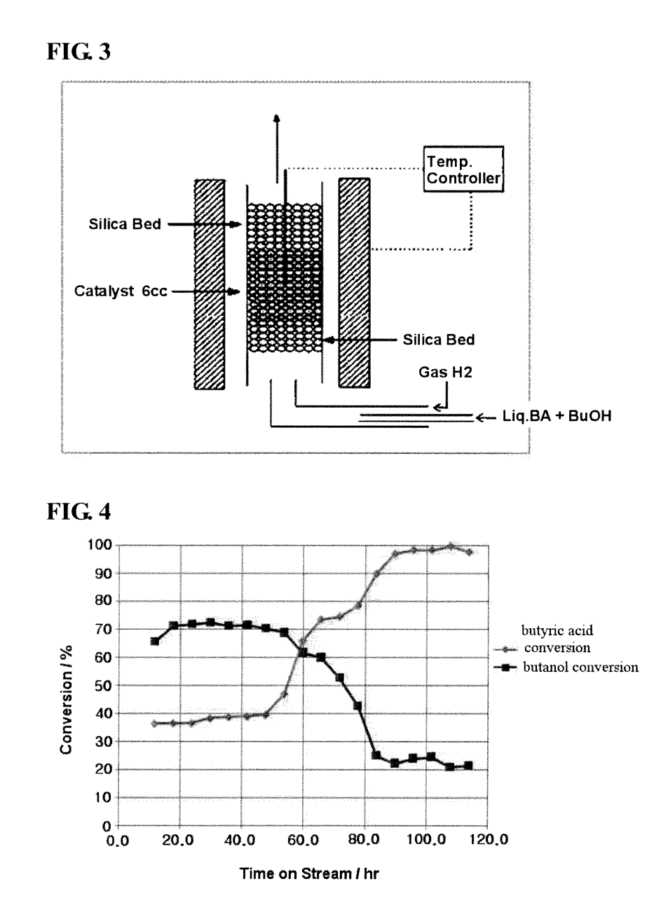

The test results are shown in FIG. 4, and the conversions at different reaction times are as follows.

(1) Initiation of reaction ˜56 hours: butanol/butyric acid (molar ratio)=0.5, flow rate of 0.2 cc/min

In consideration of the conversion, butanol and butyric acid reacted at a molar ratio of about 1:1, from which only esterification can be seen to occur.

(2) 62˜72 hours: butanol/butyric acid (molar ratio)=1.0, flow rate of 0.2 cc/min

The feed was composed of butanol and butyric acid having the same number of moles. If only esterification occurred, butanol and butyric acid had to have the same conversion, but the conversion of butyric acid was relatively higher than the conversion of butanol after 60 hours, from which it can be seen that only esterification does not occur.

(3) 78˜114 hours: butanol/butyric acid (molar ratio)=1.0, flow rate of 0.1 cc/min

The conversion of butyric acid was comparatively higher than the conversion of butanol. Further, the conversion of butyric acid was close to 100%, whereas the conversion of butanol was more than about 20%, from which normal esterification and as well hydrocracking are deemed to simultaneously occur. Specifically, butyric acid is considered to be converted into butylbutyrate by esterification and simultaneously converted into butanol by hydrocracking. Because butyric acid is converted into butanol in this way, as seen in FIG. 4, as the molar ratio of butyric acid and the residence time increase, the conversion of butyric acid is increased from 40% to 100% but the conversion of butanol is decreased from 70% to 20%, attributed to butanol converted from butyric acid.

### Example 2

### Preparation of Butanol from Butyric Acid

Butanol was prepared from butyric acid by simultaneous reactions of esterification and hydrocracking using a hydrogenation catalyst.

The reaction was performed under conditions in which the catalyst used, in-situ reduction of the catalyst just before the reaction, and the analysis method were the same as in Comparative Example 2, with the exception that a mixture of butyric acid and butanol was used, the molar ratio of butanol to butyric acid was 2.0, the flow rate was 0.1 cc/min, the molar ratio of H2 to butyric acid was 15, the reaction pressure was 10˜40 bar, and the reaction temperature was 175□.

The test results are shown in FIG. 5, and the conversions at different reaction times are given as follows.

(1) Initiation of reaction ˜88 hours: 10 bar

The butanol yield was about 58%, and the butylbutyrate yield was about 42%. Although an unknown peak was observed by GC, it was ignorable because the total area % was less than 0.2%.

(2) 94˜118 hours: 20 bar

After the pressure of the reactor was increased to 20 bar, the butanol yield was increased to about 88% whereas the butylbutyrate yield was decreased to about 12%. This is considered to be because the butanol yield is increased by the hydrocracking reaction. Specifically, although 100% esterification is carried out at both 10 bar and 20 bar, the conversion of butylbutyrate thus produced into butanol by hydrocracking is considered to accelerate at a high pressure of 20 bar.

(3) 124˜144 hours: 30 bar

After the pressure of the reactor was increased to 30 bar, the butanol yield was increased to about 95% whereas the butylbutyrate yield was further decreased to about 5%.

The reason is as described above.

(4) 150˜180 hours: 40 bar

When the pressure of the reactor was further increased to 40 bar, the results were similar to at 30 bar. The simultaneous reactions of esterification and hydrocracking are considered to reach a thermodynamic equilibrium level depending on the pressure.

(5) 186˜328 hours: 30 bar

At a reaction pressure of 30 bar which is effective, long-term stability was observed. The butanol yield was stable to the extent of about 93% for a given time, and the butylbutyrate yield represented a stable numeral of about 7%.

As shown in FIG. 5, no butyric acid was detected in the product over the entire test range. In the case where unreacted butyric acid is mixed in the product, it has a boiling point very similar to that of butylbutyrate, making it difficult to separate and purify it using simple distillation. In the present invention, there is no need to additionally purify butyric acid from the product because of butyric acid not being detected.

As shown in the above example, esterification and hydrocracking can be efficiently carried out at the same time in the one-step process according to the present invention.

### Example 3

### Preparation of Butanol from Butyric Acid at Different Temperatures

Butanol was prepared from butyric acid at different temperatures.

The reaction was performed under conditions in which the catalyst used, in-situ reduction of the catalyst just before the reaction, and the analysis method were the same as in Comparative Example 2, with the exception that a mixture of butyric acid and butanol was used, the molar ratio of butanol to butyric acid was 2.0, the flow rate was 0.1 cc/min, the molar ratio of hydrogen to butyric acid was 15, the reaction pressure was 30 bar, and the reaction temperature was 175˜250□.

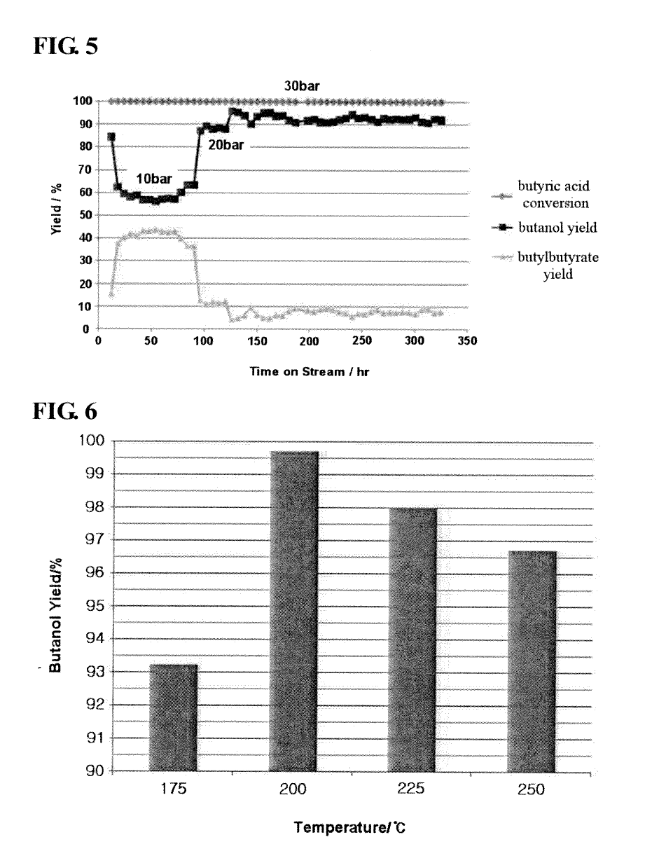

As shown in FIG. 6, a high yield of 99.7% could be obtained at 200□. At 175□, butylbutyrate, which is an esterification product, was not yet hydrocracked and was mixed in the product, and thereby the yield was slightly lowered. At 225° C. or higher, in addition to butylbutyrate, impurities were also mixed due to other side reactions. Specifically, at a temperature of less than 200□, simultaneous reactions did not sufficiently occur and thus the yield was low, whereas at a temperature exceeding 200□, other side reactions occurred in addition to the desired reaction, and reaction selectivity was decreased, undesirably lowering the yield. For this reason, 200□ is regarded as the most ideal reaction temperature.

When butanol is used as a fuel for vehicles, performance of fuel does not greatly deteriorate even in the presence of impurities such as butylbutyrate. However, when butanol is used for industrial purposes, it should have a purity of 99.5% or more. As shown in FIG. 8, the yield of 99.7% is obtained at 200° C., and thus when only moisture is removed from the product, there is no need to additionally remove impurities, and thereby butanol having high purity adapted for industrial grade can be produced.

### Example 4

### Preparation of Butanol from Butyric Acid Under Conditions of Various Reaction Gases and Different Pressures

Butanol was prepared from butyric acid under conditions of various reaction gases and different pressures.

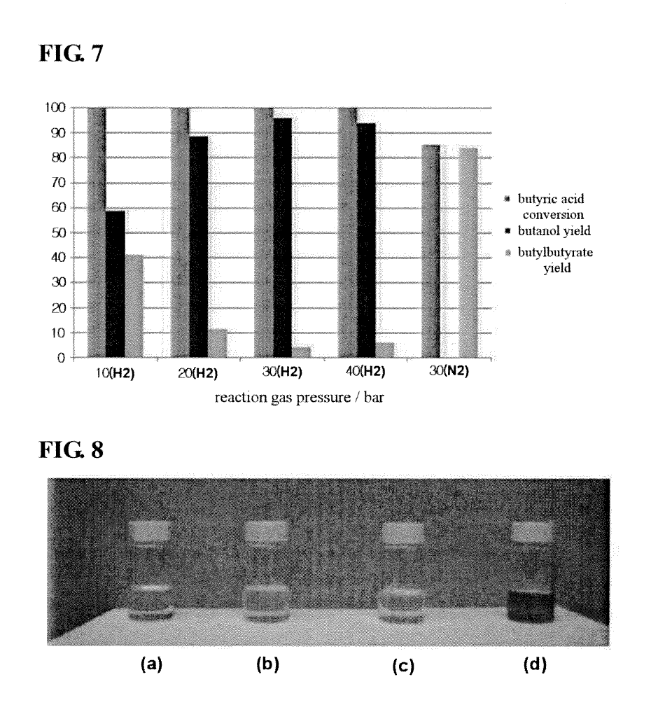

The results of Example 2 are summarized again in FIG. 7, along with the results of only esterification when hydrogen is replaced with nitrogen under the conditions of Example 2.

As shown in FIG. 7, in the case where hydrogen was replaced with nitrogen in the teed under the fixed conditions of pressure and temperature, hydrocracking did not occur and only esterification took place. As seen in FIG. 7, the butyric acid conversion was about 85%, the butylbutyrate yield was 84%, and no butanol was produced.

From such results, in the case where butyric acid, butanol, and hydrogen were reacted using a hydrogenation catalyst under given reaction conditions, esterification and hydrocracking could be seen to simultaneously occur. Also, because esterification has an equilibrium conversion, it is difficult to ensure esterification conversion of 100%. However, upon simultaneous reactions as in the present invention, butylbutyrate, which is an esterification product, was continuously removed, and the butanol feed was continuously supplied, and thus the equilibrium of esterification is shifted toward the forward reaction, thus achieving butyric acid conversion of 100%.

### Example 5

### Leaching of Catalysts Used for One-Step Process, Two-Step Process, Direct Hydrogenation (Reduction)

The reaction products resulting from hydrocracking, simultaneous reactions, and esterification using hydrogenation catalysts were compared, and the degrees of leaching of the catalysts used for respective processes were compared.

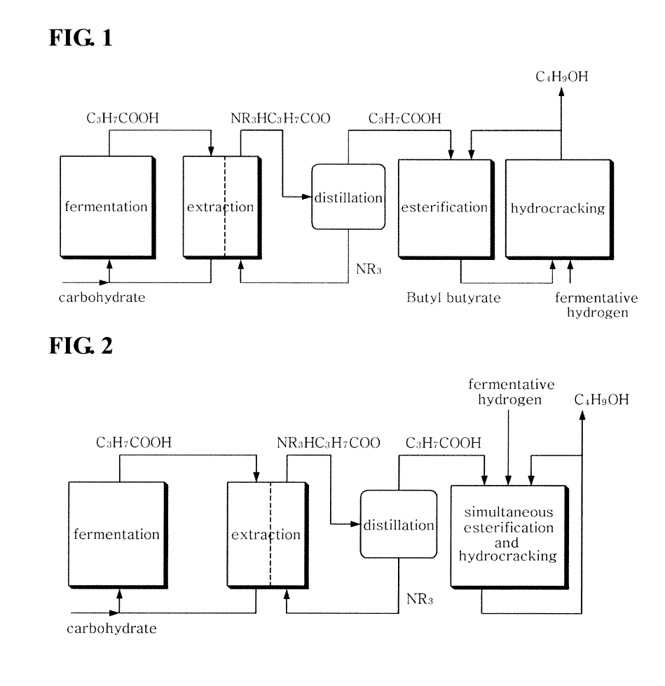

In FIG. 8, (a) shows the color of a product obtained by adding butylbutyrate as the feed of FIG. 1 and hydrocracking it, and (b) (d) show the colors of products obtained by adding hydrogen at 10 bar, hydrogen at 30 bar, and nitrogen at 30 bar respectively upon simultaneous reactions of FIG. 7, respectively.

The reaction products (a)˜(d) were subjected to ICP-AES analysis. The results are shown in Table 2 below.

TABLE 2

| | Element | | |
|---|---|---|---|
| Sample | Al | Cu | Zn |
| (a) | N/D | N/D | N/D |
| (b) | N/D | N/D | N/D |
| (c) | N/D | N/D | N/D |
| (d) | 241.0 | 171.4 | 3971 |
| Detection Limit (ppm) | 0.05 | 0.01 | 0.01 |

(a) butylbutyrate hydrocracking product (FIG. 1),
(b) simultaneous reaction product (hydrogen at 10 bar) (FIG. 7),
(c) simultaneous reaction product (hydrogen at 30 bar) (FIG. 7),
(d) simultaneous reaction product (nitrogen at 30 bar) (FIG. 7).

As is apparent from the results of ICP-AES, in the case where the hydrogenation catalyst is used for esterification, the metal component of the catalyst may be leached, seriously contaminating the product, and also the performance of the catalyst is continuously decreased, making it impossible to apply it to a catalyst for esterification.

However, in the case where the hydrogenation catalyst is used under the reaction conditions in which esterification and hydrocracking are simultaneously carried out as in the present invention, the catalyst component is not leached. This is considered to be because butyric acid, which causes leaching, is very rapidly and easily removed by esterification upon simultaneous reactions, and in contrast, in the case where only esterification occurs, unreacted butyric acid may remain behind and thus dissolves the metal component of the catalyst.

### Example 6

### Preparation of Alcohol Mixture of Butanol and Ethanol from Carboxylic Acid Mixture of Butyric Acid and Acetic Acid

An alcohol mixture of butanol and ethanol was produced by simultaneous reactions of esterification and hydrocracking from a carboxylic acid mixture of butyric acid and acetic acid using a hydrogenation catalyst.

The reaction was performed under conditions in which the catalyst used, in-situ reduction of the catalyst just before the reaction, and the analysis method were the same as in Comparative Example 2, with the exception that a mixture of butyric acid, acetic acid, and butanol was used, the molar ratio of butyric acid/acetic acid/butanol was 1:1:4, the flow rate was 0.05 cc/min, the reaction pressure was 30 bar, and the reaction temperature was 200□.

FIG. 10 shows the yields of butanol and ethanol produced from a carboxylic acid mixture of butyric acid and acetic acid. After 180 hours of the reaction time, the yield of butanol produced from butyric acid was maintained at 98% or more, and the yield of ethanol produced from acetic acid was maintained at 96% or more. Also small amounts of butylbutyrate and butylacetate were formed as byproducts.

As represented in Example 6, the one-step process according to the present invention may be applied to a carboxylic acid mixture of two or more carboxylic acids. Thus, esterification and hydrocracking were simultaneously carried out, so that an alcohol mixture of butanol and ethanol could be prepared.

Even when ethanol or an alcohol mixture of butanol and ethanol is used as the feed instead of butanol in Example 6, the production of an alcohol mixture of butanol and ethanol can be easily inferred by the same mechanism.

Similarly, in order to convert an acid mixture of two or more among acetic acid, propionic acid, butyric acid, pentanoic acid, and hexanoic acid into alcohol, in the case where one or an alcohol mixture of two or more among ethanol, propanol, butanol, pentanol, and hexanol is used as the feed, the alcohol mixture thereof can be produced by the same chemical mechanism.

As a consequence, the yield and the mixing ratio of desired alcohol may be adjusted by controlling the kind, amount, and mixing ratio of carboxylic acid and alcohol used as feeds.

### Example 7

### Continuous Production of Butyric Acid in Column-Type Fermentation Device Packed with Immobilized Strain

An anaerobic reactor for producing butyric acid from Clostridium tyrobutyricum using glucose as a carbon source was operated at 37□ on a basal medium.

In order to incubate C. tyrobutyricum at a high concentration, a column-type anaerobic reactor packed with a porous polymer carrier was used. The total volume of the reactor was 2.5 L, and the volume of packed carrier was 1.2 L.

As the polymer carrier, a sponge-type regular hexagonal porous polymer sieve composed mainly of polyurethane was used, and while glucose having a concentration of 20 g/L was continuously introduced, the concentration of produced butyric acid was measured.

5 days after C. tyrobutyricum was inoculated into the reactor, the concentration of butyric acid was increased to 8˜9 g/L. The butyric acid yield was 0.43 g butyric acid/g glucose, and the production rate of butyric acid was 6.7˜7.3 g/L-h.

The concentration of C. tyrobutyricum immobilized to the porous polymer carrier was 70 g/L or more, and desorption of the microorganism was not observed even upon continuous operation for 20 days or longer, from which C. tyrobutyricum was deemed to be stably immobilized to the porous polymer carrier, and butyric acid was stably produced a concentration of 8 g/L or more.

### Example 8

### Extraction and Distillation of Butyric Acid

Into a 500 cc cylinder, 200 g of water, 44 g of butyric acid, and 150 g of tripentylamine were added and sufficiently stirred, after which complete layer separation was achieved and then the amount of butyric acid contained in the water layer was analyzed. The concentration of butyric acid contained in the water layer was measured to be only 0.2%. Thus, 99% or more of introduced butyric acid was transferred into the tripentylamine layer, and most thereof was bonded with tripentylamine and thus converted into tripentylammonium butyrate.

175 g of the tripentylammonium butyrate layer was recovered from the cylinder, and as shown in FIG. 7, added into a batch reactor and stirred. While the pressure of the reactor was maintained at 30 ton, the inner temperature of the reactor was gradually increased at intervals of 10□ from 80□.

From the point of time at which the inner temperature of the reactor reached 90□, introduction of butyric acid vapor into the condenser was observed, and the inner temperature of the reactor was fixed to 100□.

The operation of the reactor was stopped when the butyric acid vapor introduced into the condenser was not further observed, after which 35 g of butyric acid was recovered from the receiver.

### Example 9

### Recovery of Hydrogen from Hydrogen Gas Mixture as Fermentation Byproduct

The gas mixture comprising hydrogen and carbon dioxide mixed at a molar ratio of 1:1 was separated using a pressure swing adsorption device comprising two adsorption columns packed with a zeolite adsorbent.

The operating temperature of the pressure swing adsorption device was 30□, and the operating pressure was 10 atm upon adsorption and atmospheric pressure upon desorption.

By means of the operation of the two-column pressure swing adsorption device, hydrogen having a purity of 99.9% or more could be obtained, and the total recovery rate was 83%.

Although the embodiments of the present invention have been disclosed for illustrative purposes, those skilled in the art will appreciate that a variety of different modifications, additions, and substitutions are possible without departing from the scope and spirit of the invention as disclosed in the accompanying claims. Accordingly, such modifications, additions, and substitutions should also be understood as falling within the scope of the present invention.

1. A method of preparing an alcohol, comprising reacting a carboxylic acid, an alcohol, and hydrogen, using a hydrogenation catalyst.

2. The method of claim 1, wherein the method includes esterification and hydrocracking, which are carried out in a one-step process.

3. The method of claim 1, wherein the carboxylic acid is a C2˜10 alkyl carboxylic acid, a C3˜10 cycloalkyl carboxylic acid, a C6˜10 aromatic carboxylic acid, or mixtures thereof.

4. The method of claim 1, wherein the carboxylic acid is acetic acid, propionic acid, butyric acid, pentanoic acid, hexanoic acid, or mixtures thereof.

5. The method of claim 1, wherein the carboxylic acid is obtained from a microorganism fermentation broth.

6. The method of claim 1, wherein the alcohol is a C2˜10 alcohol, a C3˜10 cycloalkyl alcohol, or a C6˜10 aromatic alcohol.

7. The method of claim 1, wherein the alcohol is ethanol, propanol, butanol, pentanol, hexanol, or alcohol mixtures thereof.

8. The method of claim 1, wherein the alcohol that is reacted with the carboxylic acid is obtained by recirculating the alcohol prepared in claim 1.

9. The method of claim 1, wherein the hydrogen that is reacted with the carboxylic acid is generated from a microorganism fermentation broth.

10. The method of claim 1, wherein a molar ratio of the alcohol to the carboxylic acid is 1.0˜50.

11. The method of claim 1, wherein the hydrogen is supplied at a molar ratio of 1˜50 to the carboxylic acid, and hydrogen pressure falls in a range of atmospheric pressure ˜100 bar.

12. The method of claim 1, wherein the hydrogenation catalyst is a metal or a metal oxide.

13. The method of claim 1, wherein the hydrogenation catalyst is one or more selected from among Cr, Mn, Fe, Co, Ni, Cu, Zn, Al, Si, Mo, W, Pt, Pd, Ru, Re, Rh, Ag, Ir, Au, and metal oxides thereof.

14. A method of preparing butanol, comprising supplying a carbohydrate so that butyric acid is fermented from a microorganism, extracting butyric acid from a fermentation broth, and reacting the extracted butyric acid with butyric acid, butanol, and hydrogen, using a hydrogenation catalyst.

15. The method of claim 14, wherein the method includes esterification and hydrocracking, which are carried out in a one-step process.

16. The method of claim 14, wherein the extracting the butyric acid from the fermentation broth comprises extracting the butyric acid using liquid-liquid extraction.

17. The method of claim 14, wherein the extracting the butyric acid further comprises distilling an extraction solvent from the extracted butyric acid.

18. The method of claim 14, wherein a molar ratio of the butanol to the butyric acid is 1.0˜50.

19. The method of claim 14, wherein the hydrogen is supplied at a molar ratio of 1˜50 to butyric acid, and hydrogen pressure falls in a range of atmospheric pressure ˜100 bar.

20. The method of claim 14, wherein the hydrogenation catalyst is a metal or a metal oxide.

21. The method of claim 14, wherein the hydrogenation catalyst is one or more selected from among Cr, Mn, Fe, Co, Ni, Cu, Zn, Al, Si, Mo, W, Pt, Pd, Ru, Re, Rh, Ag, Ir, Au, and metal oxides thereof.

22. The method of claim 3, wherein the carboxylic acid is obtained from a microorganism fermentation broth.

23. The method of claim 12, wherein the hydrogenation catalyst is one or more selected from among Cr, Mn, Fe, Co, Ni, Cu, Zn, Al, Si, Mo, W, Pt, Pd, Ru, Re, Rh, Ag, Ir, Au, and metal oxides thereof.

24. The method of claim 16, wherein the extracting the butyric acid further comprises distilling an extraction solvent from the extracted butyric acid.

25. The method of claim 15, wherein a molar ratio of the butanol to the butyric acid is 1.0˜50.

26. The method of claim 15, wherein the hydrogen is supplied at a molar ratio of 1˜50 to butyric acid, and hydrogen pressure falls in a range of atmospheric pressure ˜100 bar.

27. The method of claim 15, wherein the hydrogenation catalyst is a metal or a metal oxide.

28. The method of claim 15, wherein the hydrogenation catalyst is one or more selected from among Cr, Mn, Fe, Co, Ni, Cu, Zn, Al, Si, Mo, W, Pt, Pd, Ru, Re, Rh, Ag, Ir, Au, and metal oxides thereof.
